SEQUENCE LISTING (2 sequences)

SEQ ID NO: 1 - protein, 127 aa, synthetic construct
HVQLVESGGGSVQAGGSLRLSCAASGANGGRSCMGWFRQVPGKEREGVSGISTGGLITYYADSVKGRFTISQDNTKNTLYLQMNSLKPEDTAMYYCATSRFDCYRGSWFNRYMYNSWGQGTQVTVSS

SEQ ID NO: 2 - DNA, 375 bp, synthetic construct
  source 1..375: mol_type = other DNA
catgtgcagctggtggagtctgggggaggctcggtgcaggctggagggtctctgagactctcctgtgcagcctctggagccaacggtggtcggagctgcatgggctggttccgccaggttccagggaaggagcgcgagggggtttctggtatttcaaccggtggcttattacatactatgccgactccgtgaagggccgattcaccatctcccaagacaacaccaagaacacgctgtatctgcaaatgaacagcctgaaacctgaggacactgccatgtactagtgcgacgagtcggtttgactcgtatagaggctcttcaaccgatatatgtataaacagttggggccaggggacccaggtcactgtctcctca

(19) United States
(12) Patent Application Publication
(10) Pub. No.: US 2026/0139041 A1
(43) Pub. Date: May 21, 2026

(54) SUPPRESSION OF NEURODEGENERATIVE DISEASES BY SINGLE DOMAIN ANTIBODY

(71) Applicants: Singh Biotechnology, LLC, Clearwater, CA (US); The U.S.A., as represented by the Secretary, Department of Health and Human Services, Bethesda, MD (US)

(72) Inventors: Sunanda Singh, Clearwater, CA (US); Charles E. Egwuagu, Bethesda, MD (US); Evaristus Mbanefo, Bethesda, MD (US)

(73) Assignees: Singh Biotechnology, LLC, Clearwater, CA (US); The U.S.A., as represented by the Secretary, Department of Health and Human Services, Bethesda, MD (US)

(21) Appl. No.: 19/237,985

(22) Filed: Jan. 13, 2026

Related U.S. Application Data
(60) Provisional application No. 63/660,339, filed on Jun. 14, 2024.

(51) Int. Cl. C07K 16/18; A61K 31/137; A61K 38/17; A61K 38/21; A61K 39/00; A61K 39/395; A61K 47/26; A61P 25/28

(52) U.S. Cl. CPC C07K 16/18; A61K 31/137; A61K 38/177; A61K 38/215; A61K 39/3955; A61K 47/26; A61P 25/28; A61K 2039/505; A61K 2039/507; C07K 2317/569

(57) ABSTRACT

The present invention is directed to methods for treating or preventing neuroinflammation in a subject by administering an effective amount of a single-domain antibody (sdAb) comprising SEQ ID NO:1. The method is applicable to subjects with multiple sclerosis, including secondary progressive, primary progressive, and relapsing-remitting forms. Administration may be intravenous, subcutaneous, or intrathecal, with dosage regimens including daily administration, loading and maintenance doses, or continuous infusion. The method may be initiated upon first clinical signs of central nervous system demyelination and continued for at least 14 days. The sdAb may be co-administered with a pharmaceutically acceptable excipient such as mannitol, sucrose, or polysorbate 80, and optionally combined with disease-modifying therapies including interferon-β, glatiramer acetate, fingolimod, or ocrelizumab. The invention provides a targeted approach for modulating neuroinflammatory processes in neurological disorders.

### CROSS-REFERENCE TO RELATED APPLICATION

This application claims the benefit of U.S. Provisional Patent Application No. 63/660,339, filed on Jun. 14, 2024, the contents of which are incorporated herein by reference.

### FEDERAL FUNDING NOTICE

This invention was made with Government Support under National Institutes of Health Z01 project numbers #1ZIAEY00031533 and 1ZIAEY00035025. The United States Government has certain rights in the invention.

### SEQUENCE LISTING

The present application is being filed along with a Sequence Listing in electronic format. The Sequence Listing is provided as a file titled “Seq_Listing_ST26” created Jun. 5, 2025, and is 4,000 bytes in size. The information in the electronic format of the Sequence Listing is incorporated herein by reference in its entirety.

### BACKGROUND

The Janus kinase/signal transducer and activator of transcription (JAK/STAT) pathway is a highly conserved system that transmits signals from outside a cell to its nucleus, regulating gene expression in response to environmental cues. There are four JAKs (JAK1, JAK2, JAK3, Tyk2) and seven STAT proteins (STAT1-STAT6), with STAT1 and STAT3 playing central roles in inflammatory immune responses. When cytokines bind to receptors on lymphocytes, JAKs become activated and, in turn, activate specific STATs. These activated STATs form dimers that enter the nucleus and bind DNA to regulate gene transcription. Even inactive STATs can form dimers and influence gene expression.

Experimental autoimmune encephalomyelitis (EAE) is a mouse model of multiple sclerosis (MS), a multifocal inflammatory demyelinating disease of the central nervous system (CNS). MS can exhibit distinct clinical phenotypes depending on whether the lesions are localized to the brain, spinal cord or dispersed across CNS compartments. EAE is induced in susceptible mouse strains by active immunization with Myelin Oligodendrocyte Glycoprotein (MOG) in Complete Freund's Adjuvant (CFA) and studies using the EAE model have revealed that inflammation in the distinct microenvironments of the brain and spinal cord results from the recruitment of distinct lymphocyte populations at these CNS sites. Classic EAE (CEAE) typically affects the spinal cord and is linked to Th1 cells producing interferon-gamma (IFN-γ). Atypical EAE (aEAE) affects the brainstem and is associated with Th17 cells producing IL-17.

Because STAT1 and STAT3 are essential for the development of Th1 and Th17 cells and the inflammatory responses they drive, they are targets for MS therapies. However, since these proteins are located inside cells, targeting them with drugs is difficult. A nanobody (SBT-100) was developed that specifically targets STAT proteins. The present invention is directed towards the use of SBT-100 as a therapeutic as well as a prophylactic for MS.

### SUMMARY

The present invention provides novel methods for treating or preventing neuroinflammation in a subject in need thereof, by administering an effective amount of a single-domain antibody (sdAb) comprising SEQ ID NO:1. The disclosed methods are particularly applicable to conditions involving autoimmune-mediated central nervous system (CNS) inflammation, such as multiple sclerosis (MS), including relapsing-remitting, secondary progressive, and primary progressive forms.

In certain embodiments, the methods involve initiating treatment upon the first clinical signs of CNS demyelination and continuing for a period of at least 14 days. The sdAb comprising SEQ ID NO: 1 may be administered via intravenous, subcutaneous, or intrathecal injection, and may be delivered either as a bolus, repeated dose, or continuous infusion.

In various embodiments, effective dosages range from 0.1 to 20 mg/kg body weight per dose. Specific dosing regimens may include daily administration of 0.5 to 5 mg/kg for 10 days, an initial loading dose of 10 mg/kg followed by maintenance doses of 2 mg/kg every other day, or continuous infusion via an infusion pump at a rate of 0.5 to 2.0 mg/kg per hour. Administration may be performed with or without a pharmaceutically acceptable excipient, including but not limited to mannitol, sucrose, and polysorbate 80.

In additional embodiments, the sdAb treatment may be co-administered with one or more disease-modifying therapies commonly used in the treatment of MS. Such therapies include, for example, interferon-β, glatiramer acetate, fingolimod, and ocrelizumab.

The methods disclosed herein offer potential for both therapeutic intervention in ongoing disease as well as prophylactic application in individuals at risk for developing neuroinflammatory conditions. The use of the sdAb comprising SEQ ID NO: 1 provides a targeted, potentially more tolerable, and customizable approach to CNS immune modulation.

### DRAWINGS

These and other features, aspects, and advantages of the present invention will become better understood with regard to the following description, appended claims, and accompanying drawings where:

FIG. 1A depicts the EAE induction and SBT-100 treatment strategy; FIG. 1B depicts clinical scores of untreated EAE mice versus SBT-100 treated EAE mice; FIGS. 1C and 1D depicts plots showing histopathology score in of untreated versus treated EAE mice in the brain and spinal cord; FIG. 1E shows a three day [3H]-thymidine incorporation assay;

FIGS. 2A and 2B depicts the gating strategy for CD4+ T cells for FACS plots;

FIGS. 3A-3E depicts plots showing histopathology scores in untreated versus treated mice in lymphocytes isolated from the brain, spinal cord, lymph nodes (LN) or spleen and subjected to intracellular cytokine staining analysis;

FIGS. 4A-4G depicts graphs showing cells from the lymph nodes and spleen of control or SBT-100-treated mice with EAE that were re-stimulated with MOG35-55 for 3 days followed by transfer to unimmunized naive syngeneic mice; and

FIG. 5 depicts a frequency plot of intracellular cytokine staining and FACS analysis showing the number of IL-17A+, IFN-γ+, and IL-17A+IFN-γ+ double positive cells in the spinal cord.

### DESCRIPTION

As used herein, the following terms and variations thereof have the meanings given below, unless a different meaning is clearly intended by the context in which such term is used.

The terms “a,” “an,” and “the” and similar referents used herein are to be construed to cover both the singular and the plural unless their usage in context indicates otherwise.

The term “antigenic determinant” refers to the epitope on the antigen recognized by the antigen-binding molecule (such as an sdAb or polypeptide of the invention) and more in particular by the antigen-binding site of the antigen-binding molecule. The terms “antigenic determinant” and “epitope” may also be used interchangeably. An amino acid sequence that can bind to, that has affinity for and/or that has specificity for a specific antigenic determinant, epitope, antigen or protein is said to be “against” or “directed against” the antigenic determinant, epitope, antigen or protein.

As used herein, the term “comprise” and variations of the term, such as “comprising” and “comprises,” are not intended to exclude other additives, components, integers or steps.

It is contemplated that the sdAbs, polypeptides and proteins described herein can contain so-called “conservative” amino acid substitutions, which can generally be described as amino acid substitutions in which an amino acid residue is replaced with another amino acid residue of similar chemical structure, and which has little or essentially no influence on the function, activity or other biological properties of the polypeptide. Conservative amino acid substitutions are well known in the art. Conservative substitutions are substitutions in which one amino acid within the following groups (a)-(e) is substituted by another amino acid within the same group: (a) small aliphatic, nonpolar or slightly polar residues: Ala, Ser, Thr, Pro and Gly; (b) polar, negatively charged residues and their (uncharged) amides: Asp, Asn, Glu and Gln; (c) polar, positively charged residues: His, Arg and Lys; (d) large aliphatic, nonpolar residues: Met, Leu, Ile, Val and Cys; and (e) aromatic residues: Phe, Tyr and Trp. Other conservative substitutions include: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

A “domain” as used herein generally refers to a globular region of an antibody chain, and in particular to a globular region of a heavy chain antibody, or to a polypeptide that essentially consists of such a globular region.

The amino acid sequence and structure of an sdAb is typically made up of four framework regions or “FRs,” which are referred to as “Framework region 1” or “FR1”; as “Framework region 2” or “FR2”; as “Framework region 3” or “FR3”; and as “Framework region 4” or “FR4,” respectively. The framework regions are interrupted by three complementarity determining regions or “CDRs,” which are referred as “Complementarity Determining Region 1” or “CDR1”; as “Complementarity Determining Region 2” or “CDR2”; and as “Complementarity Determining Region 3” or “CDR3,” respectively.

As used herein, the term “humanized sdAb” means an sdAb that has had one or more amino acid residues in the amino acid sequence of the naturally occurring VHH sequence replaced by one or more of the amino acid residues that occur at the corresponding position in a VH domain from a conventional 4-chain antibody from a human. This can be performed by methods that are well known in the art. For example, the FRs of the sdAbs can be replaced by human variable FRs.

As used herein, an “isolated” nucleic acid or amino acid has been separated from at least one other component with which it is usually associated, such as its source or medium, another nucleic acid, another protein/polypeptide, another biological component or macromolecule or contaminant, impurity or minor component.

The term “mammal” is defined as an individual belonging to the class Mammalia and includes, without limitation, humans, domestic and farm animals, and zoo, sports, and pet animals, such as cows, horses, sheep, dogs and cats.

As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field. Preferred examples of such carriers or diluents include, but are not limited to, water, saline, Ringer's solutions, dextrose solution, PBS (phosphate-buffered saline), and 5% human serum albumin. Liposomes, cationic lipids and non-aqueous vehicles such as fixed oils may also be used. The use of such media and agents for pharmaceutically active substances is well known in the art. Except insofar as any conventional media or agent is incompatible with a therapeutic agent as defined above, use thereof in the composition of the present invention is contemplated.

A “quantitative immunoassay” refers to any means of measuring an amount of antigen present in a sample by using an antibody. Methods for performing quantitative immunoassays include, but are not limited to, enzyme-linked immunosorbent assay (ELISA), specific analyte labeling and recapture assay (SALRA), liquid chromatography, mass spectrometry, fluorescence-activated cell sorting, and the like.

The term “solution” refers to a composition comprising a solvent and a solute, and includes true solutions and suspensions. Examples of solutions include a solid, liquid or gas dissolved in a liquid and particulates or micelles suspended in a liquid.

The term “specificity” refers to the number of different types of antigens or antigenic determinants to which a particular antigen-binding molecule or antigen-binding protein molecule can bind. The specificity of an antigen-binding protein can be determined based on affinity and/or avidity. The affinity, represented by the equilibrium constant for the dissociation of an antigen with an antigen-binding protein (KD), is a measure for the binding strength between an antigenic determinant and an antigen-binding site on the antigen-binding protein: the lesser the value of the KD, the stronger the binding strength between an antigenic determinant and the antigen-binding molecule (alternatively, the affinity can also be expressed as the affinity constant (KA), which is 1/KD). As will be clear to one of skill in the art, affinity can be determined depending on the specific antigen of interest. Avidity is the measure of the strength of binding between an antigen-binding molecule and the antigen. Avidity is related to both the affinity between an antigenic determinant and its antigen binding site on the antigen-binding molecule and the number of pertinent binding sites present on the antigen-binding molecule. Specific binding of an antigen-binding protein to an antigen or antigenic determinant can be determined by any known manner, such as, for example, Scatchard analysis and/or competitive binding assays, such as radioimmunoassays (RIA), enzyme immunoassays (EIA) and sandwich competition assays.

As used herein, the term “recombinant” refers to the use of genetic engineering methods (for example, cloning, and amplification) used to produce the sdAbs of the invention.

A “single domain antibody,” “sdAb” or “VHH” can be generally defined as a polypeptide or protein comprising an amino acid sequence that is comprised of four framework regions interrupted by three complementarity determining regions. This is represented as FR1-CDR1-FR2-CDR2-FR3-CDR3-FR4. An sdAb of the invention also includes a polypeptide or protein that comprises the sdAb amino acid sequence. Typically, sdAbs are produced in camelids such as llamas, but can also be synthetically generated using techniques that are well known in the art. As used herein, the variable domains present in naturally occurring heavy chain antibodies will also be referred to as “VHH domains,” in order to distinguish them from the heavy chain variable domains that are present in conventional 4-chain antibodies, referred to as “VH domains,” and from the light chain variable domains that are present in conventional 4-chain antibodies, referred to as “VL domains.” “VHH” and “sdAb” are used interchangeably herein. The numbering of the amino acid residues of a sdAb or polypeptide is according to the general numbering for VH domains given by Kabat et al. (“Sequence of proteins of immunological interest,” US Public Health Services, NIH Bethesda, MD, Publication No. 91). According to this numbering, FR1 of a sdAb comprises the amino acid residues at positions 1-30, CDR1 of a sdAb comprises the amino acid residues at positions 31-36, FR2 of a sdAb comprises the amino acids at positions 36-49, CDR2 of a sdAb comprises the amino acid residues at positions 50-65, FR3 of a sdAb comprises the amino acid residues at positions 66-94, CDR3 of a sdAb comprises the amino acid residues at positions 95-102, and FR4 of a sdAb comprises the amino acid residues at positions 103-113.

The term “synthetic” refers to production by in vitro chemical or enzymatic synthesis.

The term “target” as used herein refers to any component, antigen, or moiety that is recognized by the sdAb. The term “intracellular target” refers to any component, antigen, or moiety present inside a cell. A “transmembrane target” is a component, antigen, or moiety that is located within the cell membrane. An “extracellular target” refers to a component, antigen, or moiety that is located outside of the cell.

A “therapeutic composition” as used herein means a substance that is intended to have a therapeutic effect such as pharmaceutical compositions, genetic materials, biologics, and other substances. Genetic materials include substances intended to have a direct or indirect genetic therapeutic effect such as genetic vectors, genetic regulator elements, genetic structural elements, DNA, RNA and the like. Biologics include substances that are living matter or derived from living matter intended to have a therapeutic effect.

As used herein, the phrases “therapeutically effective amount” and “prophylactically effective amount” refer to an amount that provides a therapeutic benefit in the treatment, prevention, or management of a disease or an overt symptom of the disease. The therapeutically effective amount may treat a disease or condition, a symptom of disease, or a predisposition toward a disease, with the purpose to cure, heal, alleviate, relieve, alter, remedy, ameliorate, improve, or affect the disease, the symptoms of disease, or the predisposition toward disease. The specific amount that is therapeutically effective can be readily determined by an ordinary medical practitioner, and may vary depending on factors known in the art, such as, e.g., the type of disease, the patient's history and age, the stage of disease, and the administration of other therapeutic agents.

The present invention relates to the use of single-domain antibodies (sdAbs) that are directed against intracellular components, as well as to the use of proteins and polypeptides comprising the sdAbs and nucleotides encoding the proteins and polypeptides to treat or prevent disease. The invention also includes nucleic acids encoding the sdAbs, proteins and polypeptides, and compositions comprising the sdAbs.

The amino acid sequence of an anti-STAT sdAb, named SBT-100 (or VHH13) (SEQ ID NO:1), is shown below, with the three CRDs underlined:

HVQLVESGGGSVQAGGSLRLSCAASGANGGRSCMGWFRQVPGKEREGV
SGISTGGLITYYADSVKGRFTISQDNTKNTLYLQMNSLKPEDTAMYYC
ATSRFDCYRGSWFNRYMYNSWGQGTQVTVSS

The corresponding anti-STAT3 SBT-100 DNA sequence (SEQ ID NO:2) is:

5′catgtgcagctggtggagtctgggggaggctcggtgcaggctggag
ggtctctgagactctcctgtgcagcctctggagccaacggtggtcgga
gctgcatgggctggttccgccaggttccagggaaggagcgcgaggggg
tttctggtatttcaac-catgggctggttccgccaggttccagggaag
gagcgcgagggggtttctggtatttcaac-cggtggtcttattacata
ctatgccgactccgtgaagggccgattcaccatctcccaaga-caaca
ccaagaacacgctgtatctgcaaatgaacagcctgaaacctgaggaca
c-tgccatgtactactgtgcgacgagtcggtttgactgctatagaggc
tcttggttcaaccgata-tatgtataacagttggggccaggggaccca
ggtcactgtctcctca-3′

SBT-100 (SEQ ID NO:1) has been previously described in U.S. patent Ser. No. 14/922,093, the contents of which are incorporated herein by reference. Briefly, SBT-100 (SEQ ID NO:1) is a nanobody, or single domain antibody (sdAb), consisting of a single VHH derived from a camelid immunoglobulin heavy chain variable region devoid of light chain. SBT-100 (SEQ ID NO:1) can penetrate lymphocytes and inhibits IL-6/STAT3 signaling pathways of primary mouse CD4+ lymphocytes and human Jurkat T-cells. The use of single-domain antibodies (sdAbs) as single antigen-binding proteins or as an antigen-binding domain in larger protein or polypeptide offers a number of significant advantages over the use of conventional antibodies or antibody fragments. The advantages of sdAbs include: only a single domain is required to bind an antigen with high affinity and with high selectivity; sdAbs can be expressed from a single gene and require no post-translational modification; sdAbs are highly stable to heat, pH, proteases and other denaturing agents or conditions; sdAbs are inexpensive to prepare; and sdAbs can access targets and epitopes not accessible to conventional antibodies.

Since the sdAbs of the invention are mainly intended for therapeutic use, they are directed against mammalian, preferably human, targets. However, it is possible that the sdAbs described herein are cross-reactive with targets from other species, for example with targets from one or more other species of primates or other animals (for example, mouse, rat, rabbit, pig or dog), and in particular animal models for diseases and disorders associated with the disease associated with the targets.

The invention further relates to applications and uses of the sdAb, the nucleic acids encoding the sdAbs, host cells, products and compositions described herein. Such a product or composition may, for example, be a pharmaceutical composition for treatment or prevention of a disease.

The present invention generally relates to sdAbs, as well as to proteins or polypeptides comprising or essentially consisting of one or more of such sdAbs, that can be used for prophylactic and therapeutic purposes.

The methods and compositions detailed in the present invention can be used to prevent or treat the diseases described herein, and can be used with any dosage and/or formulation described herein or otherwise known, as well as with any route of administration described herein or otherwise known to one of skill in the art.

The sdAb of the invention can be used with one or more compounds. The one or more compounds can increase the therapeutic response and augment the effectiveness of the sdAb of the invention. In addition, the effectiveness of the sdAb can be increased by combining it with peptides, peptidomimetics, and other drugs.

SBT-100 (SEQ ID NO:1) has also been shown to be effective in suppressing inflammation in vivo using experimental autoimmune uveitis induced in C57BL/6J mice by active immunization with the ocular autoantigen, interphotoreceptor retinoid binding protein (IRBP). Analysis of the retina by fundoscopy, histological examination or optical coherence tomography showed that the treatment of mice with SBT-100 (SEQ ID NO:1) suppressed uveitis by inhibiting expansion of pathogenic Th17 cells that mediate EAU. Electroretinographic (ERG) recordings of dark and light adapted a- and b-waves showed that SBT-100 (SEQ ID NO:1) treatment prevented the mice from developing significant visual impairment that characterize EAU in untreated mice. Adoptive transfer of activated IRBP-specific T-cells from untreated EAU mice induced EAU, while EAU was significantly attenuated in mice that received IRBP-specific T-cells from SBT-100 (SEQ ID NO:1) treated mice. (U.S. patent application Ser. No. 17/859,861). The results show that SBT-100 (SEQ ID NO:1) can cross the BRB, treat an organ specific autoimmune ophthalmic disease, and significantly inhibit both TH17 and TH1 cells in vivo.

Central nervous system (CNS) autoimmune diseases such as uveitis and multiple sclerosis (MS) result as consequence of breakdown of immune privilege of the brain, spinal cord or neuroretina which are maintained by the blood-retina barrier (BRB), blood-brainbarrier (BBB) and the neurovascular unit (NVU) comprised of pericytes, perivascular macrophages, tightly bound endothelial cells, glia limitans of the Müller/microglia. These structures sequester CNS tissues from peripheral immune system and Th17 cells that produce Granzyme B are implicated in early events that initiate CNS autoimmune diseases by promoting the disruption of the BBB or BRB. However, sustained activation of microglial cells and recruitment of other inflammatory cells amplify the inflammatory response and are responsible for pathology characteristic of chronic uveitis or multiple sclerosis (MS). Nonetheless, interventional studies using biologics such as cytokines or immune-suppressive compounds to suppress uveitis in mice invariably show strong correlation of disease amelioration with suppression of pathogenic Th17 cells. Subsequent studies revealed the requirement of STAT3 for Th17 differentiation and development while others showed that targeted deletion of STAT3 prevented the development of EAE or EAU. These studies led to the now established notion that targeting Th17 cells is a viable therapeutic approach for suppressing and mitigating autoimmune and autoinflammatory diseases.

MS is a multifocal inflammatory demyelinating disease of the CNS. In some patients the disease is restricted to the spinal cord, while in others it presents in the brain or other CNS compartments. Although the etiology of MS is not well understood, specific T cell subsets have been identified in histopathological lesions of mice with EAE as well as MS patients, but it is now well established that inflammation is regulated differently in the brain and spinal cord. The secretion of IFN-γ is implicated in the disease characterized by ascending paralysis while IL-17 secretion mediates inflammation of the cerebellum including ataxia. Consequently, therapeutic strategies that specifically target Th1 responses that mediate spinal cord inflammation may not be effective for suppressing cerebellar ataxia caused by Th17 cells.

There is currently no cure for MS. However, corticosteroids (oral prednisone and intravenous methylprednisolone) are effective in suppressing relapsing-remitting MS and the therapeutic goal is to promote rapid recovery from attacks or slow down progression of the disease. However, side effects of corticosteroids can increase blood pressure, induce elevated glucose, and cause fluid retention. Besides steroids, treatments that modify the progression of relapsing-remitting MS are also in use and the most prescribed drug among the disease modifying therapies (DMTs) is Interferon beta (IFN-beta). Side effects of IFN-beta treatment also have adverse effects including liver damage and reduced drug efficacy due to development of neutralizing antibodies. The significant adverse effects of these immunosuppressive therapies are the impetus to develop alternative therapies MS. Effective therapy for MS must therefore be designed to suppress inflammation in both the spinal cord (mediated by IFN-γ signaling) and the brain (Th17/IL-17) because specific targeting of T cells in either microenvironment may be ineffective.

As shown in the Examples below, SBT-100 (SEQ ID No. 1) nanobody is effective in suppressing EAE by inhibiting the STAT3 pathway and inflammatory responses mediated by Th17 cells in the brain. SBT-100 also suppresses the STAT1 signaling pathway that is required for IFN-γ signaling and Th1 immunological responses in the spinal cord.

The JAK/STAT pathway plays a critical role in regulating cytokines including IL-6, IL-12. IL-23, IFN-γ, and GM-CSF implicated in MS. Consequently, there has been interest in targeting JAK/STAT pathway as treatment of MS or EAE. For example, Baricitinib, a JAK 1/2 inhibitor and AZD1480, another JAK1/2 inhibitor have been effective in ameliorating EAE. On the other hand, Tofacitinib inhibits JAK1, JAK2, JAK3 and is approved by the Food and Drug Administration (FDA) for treating rheumatoid arthritis (RA), psoriatic arthritis and ulcerative colitis. However, it has shown contradictory effects on multiple sclerosis in animal models. Moreover, use of Tofacitinib induced iatrogenic multifocal CNS demyelination in a RA patient. Despite the interest in JAK kinase as treatment for MS, few studies have focused on the therapeutic use of STAT inhibitors. In the Examples below, SBT100 suppressed the expansion of Th1 and Th17 in the brain and spinal cord and mice treated with SBT-100 were protected from severe encephalomyelitis. Moreover, the relatively small size of the miniature SBT-100 nanobody facilitates its entry into the CNS, suggesting that SBT-100 immunotherapy can be exploited as a safe therapeutic option for treatment of neuroinflammatory diseases such as MS.

### EXAMPLES

### Example 1: Characterization of the SBT-100 Nanobody

The STAT family of proteins are latent cytoplasmic transcription factors that are comprised of six domains: the N-terminal domain, coiled-coil domain, DNA binding domain, linker domain, SH2 domain and Trans activation domain. Binding of cytokines or growth factors to cognate receptors on lymphocytes activate JAKs resulting in selectively recruitment and activation of requisite STATs that transduce cytokine/growth factor signals to the nucleus. A STAT-specific nanobody, SBT-100 (SEQ ID NO. 1), was found to suppress experimental autoimmune uveitis (EAU), an autoimmune disease that serves as mouse model of human uveitis (U.S. patent application Ser. No. 17/859,861). Although EAU is mediated by Th17 cells, SBT-100 suppressed Th1 and Th17 cells but had no effect on the Treg subset that requires Foxp3 for its differentiation and development. Previous results showed that SBT-100 inhibits STAT3 pathway of uveitogenic Th17 and Th1 cells that mediate uveitis (U.S. patent application Ser. No. 17/859,861).

### Example 2: SBT-100 Ameliorates EAE by Suppressing Inflammation in the Brain and Spinal Cord

Six- to eight-week-old female C57BL/6J mice were purchased from Jackson Laboratory (Jackson Laboratory, Bar Harbor, ME). Animals were housed at the National Institutes of Health (NIH)/National Eye Institute (NEI) animal facility, maintained under 12-hour light-dark cycle with unlimited access to water and chow, and provision of nutritional supplements at onset of disease. All animal care and procedures were humane and conformed with the National Institute of Health Animal Care and Use Committee guidelines. The experiments were approved and performed under the NIH/NEI Animal Study Protocol (ASP #NEI597).

Experimental Autoimmune Encephalitis (EAE) was induced by subcutaneous immunization of C57BL/6J mice with 200 μg myelin oligodendrocyte glycoprotein peptide (MOG₃₅₋₅₅) (Sigma, ST Louis, MO) in CFA emulsion, containing 4.0 mg/ml of heat killed, pulverized Mycobacterium tuberculosis strain H37RA. The mice also received two doses of 0.3 μg Bordetella pertussis toxin (Sigma, St. Louis, MO) on day 0, and day 2 postimmunization by intraperitoneal (i.p.) injection in 100 μl of PBS containing 1.0% normal mouse serum. Starting from Day 0 of immunization to Day 12 post EAE induction, mice were treated twice daily with 100 μl PBS (untreated group) or SBT-100 (10 mg/kg body weight in 100 μl PBS). Previous studies established that administering 10 mg/kg SBT-100 intraperitoneally is effective in suppressing inflammation in the retina (U.S. patent application Ser. No. 17/859,861). Because the retina is also a CNS tissue, this dosing regimen was used. Disease progression was assessed every 2 days and clinical symptoms of EAE were graded according to Table 1 below:

TABLE 1
EAE Scoring

0, no clinical symptoms
0.25, 25% of tail flaccid
0.5, 50% of tail flaccid
0.75, 75% of tail flaccid
1.0, limp flaccid tail
then in addition to flaccid tails
1.5, paraparesis of 1 hindlimb
2.0, paraparesis of 2 hindlimb
2.5, paralysis of 1 hindlimb
3.0, paralysis of 2 hindlimb
3.5, paralysis of 2 hindlimb and weakness of 1 forelimb
4.0, paralysis of 2 hindlimb and weakness of 2 forelimb
4.5, complete hindlimbs and forelimbs paralysis
5.0, complete hindlimbs and forelimbs paralysis and reduced
responsiveness to external stimulation (sacrifice indicated).

For histopathological examination, spinal cord and brain were harvested on Day 21 post immunization, fixed using formaldehyde, dehydrated in ethanol, and sent for paraffin embedding and sectioning by HistoServ Inc or NEI Imaging core facility. Sections (5 μm) were stained with hematoxylin and eosin (H&E) or used for immunohistochemistry. The slides were deparaffinized and processed for antigen retrieval in Tris EDTA buffer (pH 9.0) using HIER DECLOAKING CHAMBER™ (Biocare, Pacheco, CA) for 15 min at 110° C. followed by slow cooling. Non-specific reactions were blocked using 10% serum, including 1% bovine serum albumin (BSA), 3% skimmed milk, 0.02% sodium azide, and 0.1% Triton X100. The primary antibody, Rabbit Anti-Mouse CD4 (Ab183685, Abcam, Boston, MA), was incubated overnight at 4° C. in the blocking buffer. The secondary antibody Goat Anti-Rabbit AF568 (A11036, Invitrogen) was incubated for 1 h at RT. The slides were washed and mounted in the EverBrite TrueBlack® Hardset Mounting Medium with DAPI (Biotium, Fremont, CA). Images were acquired using a Zeiss Axio Observer 7 (Zeiss, Oberkochen, Germany). The images acquired using 10× objectives were stitched together by Zeiss ZenBlue-Edition software (Zeiss, Oberkochen, Germany), and processed using Imaris 9.9.0.

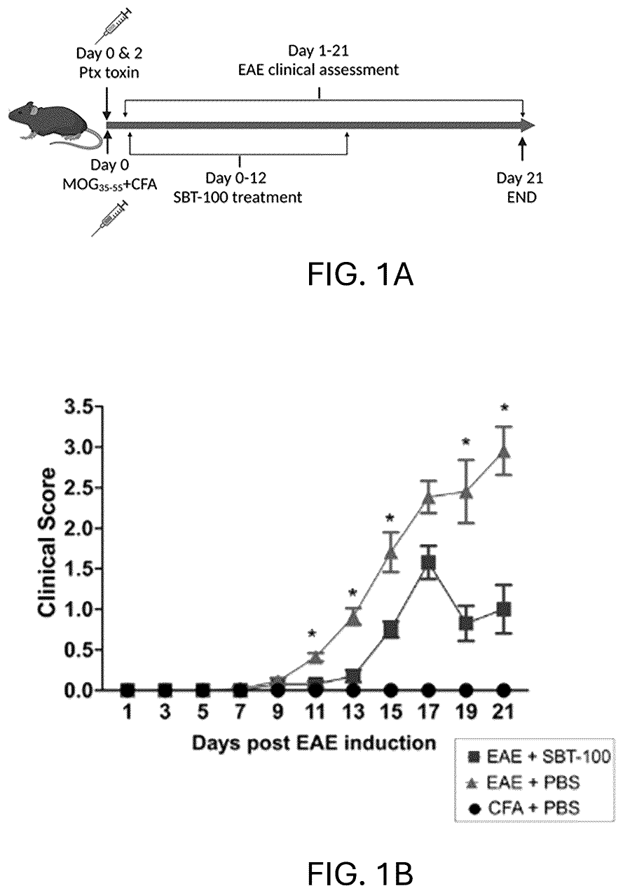

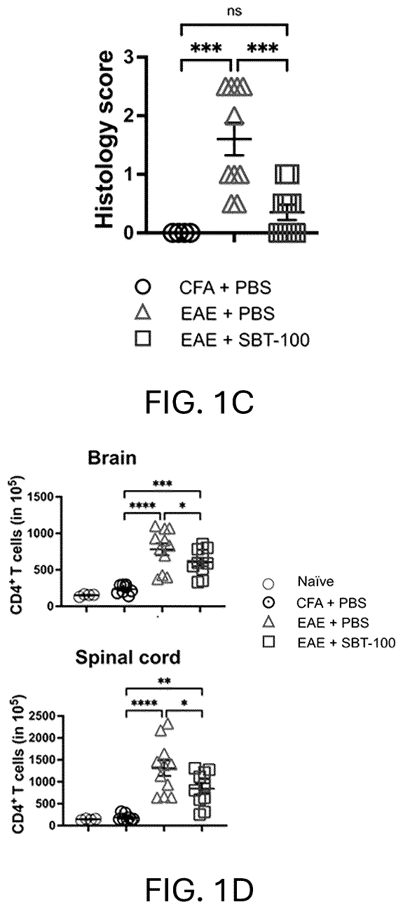

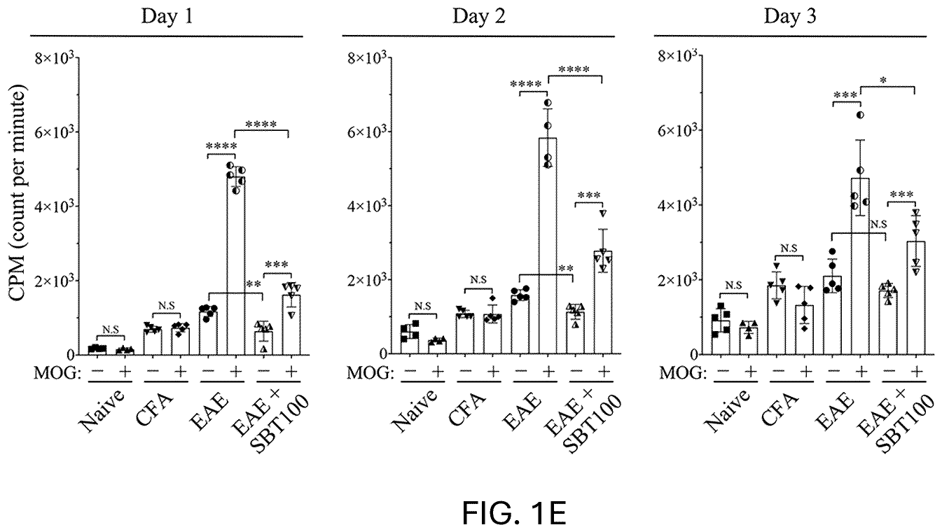

The treatment group was treated with SBT-100 nanobody from Day 0 to Day 12 post EAE induction as indicated by the AEA induction and SBT-100 treatment strategy detailed in FIG. 1A. Control mice treated with PBS developed pathognomonic features of EAE including: infiltration of inflammatory cells into the brain and spinal cord, development of flaccid tail or front/hind limb paralysis. Disease scores assessed by masked investigators. Representative clinical scores of untreated EAE mice versus SBT-100 treated EAE mice show reduced EAE symptoms in SBT-100 treated mice (blue square), n=10-12. As the disease progressed, some mice became moribund while these hallmark features of EAE were attenuated in SBT-100-treated mice as indicated by reduced EAE disease scores (FIG. 1B).

Immunohistochemical scoring showed significant increase of CD4+ T cells in the spinal cord of control mice compared to SBT-100 treated mice. Representative histopathology score of untreated EAE mice versus SBT-100 treated EAE mice are shown in FIG. 1C. Compared to SBT-100 treated mice, immunohistochemical analysis of Day 21 spinal cord revealed increased infiltration of inflammatory cells into the CNS tissues of the untreated mice which corresponded to higher histology scores.

On day 21 post-immunization, CD4+ T cells were isolated and quantified from the brain or spinal cord (n=10-12). Significantly reduced numbers of T cells in the brain and spinal cord of SBT-100 treated mice were observed (FIG. 1D).

To assess the proliferation of encephalitogenic T cells, draining lymph node cells were seeded at a concentration of 2×10⁶/mL and restimulated with MOG35-55-peptide (20 μg/ml) for 24, 48 and 72 hours with or without different concentrations of SBT-100 (100 μg/ml, 50 μg/ml, 25 μg/ml or 12.5 μg/ml for some experiments). The cells were pulsed with [3H]-thymidine (0.5 μCi/10 μl/well) during the last 12 hours of each culture timepoints. Presented data are mean count per minute (CPM)±SD of responses of 6 replicate cultures.

[3H]-thymidine incorporation assay was preformed, showing that SBT-100 inhibits proliferation of MOG-specific encephalitogenic T cells. T cells were isolated on day 21 post-immunization were restimulated in vitro with MOG₃₅₋₅₅ for 3 days and the effect of SBT-100 on the proliferation of the MOG-specific encephalitogenic T cells were quantified by the lymphocyte proliferation assay. As shown in FIG. 1E, at each time point analyzed, there was a significant decrease in the proliferative capacity of T cells stimulated in medium containing SBT-100 compared to control cells that received PBS were observed, as shown by the [3H]-thymidine incorporation assay. This indicates that SBT-100 inhibits proliferation of MOG-specific encephalitogenic T cells (n=5). Data represents at least 2 independent experiments and is presented as mean±SEM. (*p<0.05; **p<0.01; ***p<0.001; ****p<0.0001). Interestingly, SBT-100 seems to reduce T cell proliferation without MOG.

These results indicate that SBT-100 suppresses the expansion of encephalitogenic T cells and that mitigation of EAE by SBT-100 immunotherapy derived in part from SBT100-mediated reduction of encephalitogenic T cells and proinflammatory responses that induce inflammation in the brain and spinal cord.

### Example 3. SBT-100 Mediated Targeting of STATs is Effective in Suppressing EAE

IFN-γ-producing Th1 lymphocytes and IL-17-producing Th17 cells play critical roles in development of EAE in the brain and spinal cord, and commitment to their respective developmental pathway and phenotype requires sustained activation of STAT1 or STAT3 signal transduction pathway, respectively. Studies were done to examine whether the suppression of EAE in mice treated with SBT-100 was derived in part from inhibition of STAT1 and STAT3 pathways that induce development of Th1 and Th17 cells or from suppression of effector functions of IFN-γ and IL-17 in the brain or spinal cord. CD4⁺ T cells were isolated from the brain and spinal cord as well as the spleen and draining lymph nodes and analyzed by the intracellular cytokine staining assay.

For intracellular cytokine detection, cells were re-stimulated for 5 h with PMA (50 ng/ml) and ionomycin (500 ng/ml). GolgiPlug (BD Pharmingen, San Diego, CA) was added in the last 2 hour, and intracellular cytokine staining was performed using the BD Biosciences Cytofix/Cytoperm kit as recommended. Dead cells were stained with live/dead Fixable Dead Cell Stain Kits (Invitrogen, Carlsbad, CA).

FACS analysis was performed on cells stained with fluorescent labeled monoclonal antibodies specific to intracellular cytokines and transcription factors or corresponding isotype antibodies. Dead cells were excluded, and each tube of cells was color-compensated. Quadrant gates were set using isotype controls with less than 0.5% background. FACS analysis was performed on CytoFLEX Flow Cytometer (Beckman Coulter, Indianapolis, IN). Some samples were analyzed using Cytek Aurora System (Cytek, Bethesda, MD). Data analysis was performed on FlowJo version 10.9.0.

To prepare single cell suspension of CNS tissues, draining lymph nodes and spleen, mice were euthanized and extensively perfused with PBS before lymphoid and CNS tissues were aseptically excised. CNS infiltrated lymphocytes/mononuclear cells were collected from the brain and spinal cord by gentle dissociation using gentle MACS™ (Miltenyi Biotec, Auburn, CA, Cat #130-093-235), digested with Collagenase (1 mg/ml) and DNase (10 μg/ml) and subjected to Percoll gradient centrifugation (Cat #17089101, Cytiva, Uppsala, Sweden). Centrifuged cells were resuspended in 30% Percoll and layered on 70% Percoll followed by centrifugation at 2500 rpm at RT for 25 min. The cells at the 30% and 70% Percoll interface were collected, washed twice, strained through 40 μm cell strainer, and counted using the Vi-Cell XR cell viability analyzer (Beckman Coulter, Brea, CA). draining lymph nodes and spleens were dissected, and cells freed by teasing in a 40 μm pore cell strainer. Washed cells were suspended in RPMI 1640 medium, erythrocytes lysed in ACK RBC lysis buffer (Quality Biological, Gaithersburg, MD) and lysis was terminated in 10× volume of the medium. Cells were washed (2×), resuspended in medium and seeded at a concentration of 2× 10⁶/ml.

Data analysis and graph plots were performed on GraphPad Prism 9, using two-tailed unpaired Student's t test for pairwise comparisons. Two-way ANOVA was performed for each time point of the clinical scores. For multiple comparisons, One-way ANOVA with multiple pairwise t tests were performed. Data are representative of at least 2 independent experiments and are shown as mean and SEM and statistical significance for inferences was based on p<0.05. Asterisks in figures denote p-values (*p<0.05, **p<0.01, ***p<0.001, ****p<0.0001).

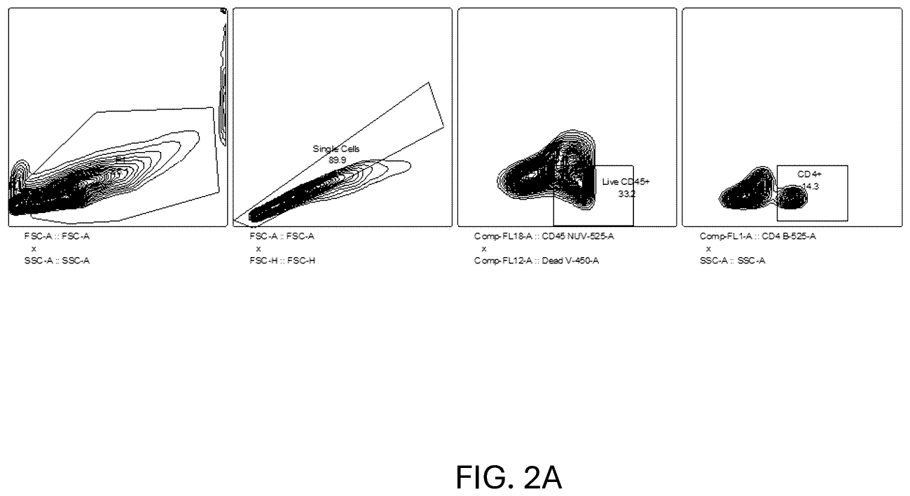

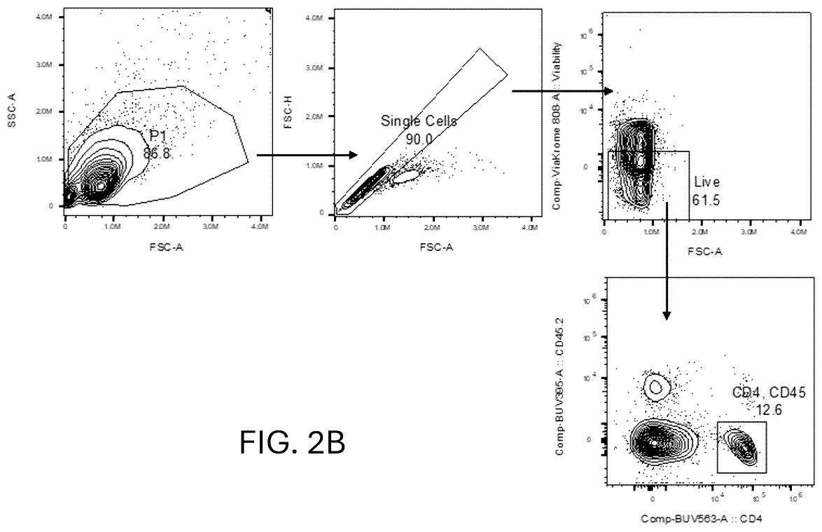

The CD4+ T cell representative gating strategy is shown in FIG. 2A. SBT-100 suppresses encephalomyelitis by inhibiting pathogenic Th17 and Th1 cells. Lymphocytes isolated from the brain, spinal cord, lymph nodes (LN) or spleen of EAE mice treated with PBS or SBT-100 were subjected to intracellular cytokine staining analysis. Flow cytometry plots indicate percentage of T cells in the brain or spinal cord expressing IL-17 and/or IFN-γ. Cytometry percentage bar graphs show suppression of T cells expressing IL-17 and/or IFN-γ in the brain and spinal cord of mice treated with SBT-100.

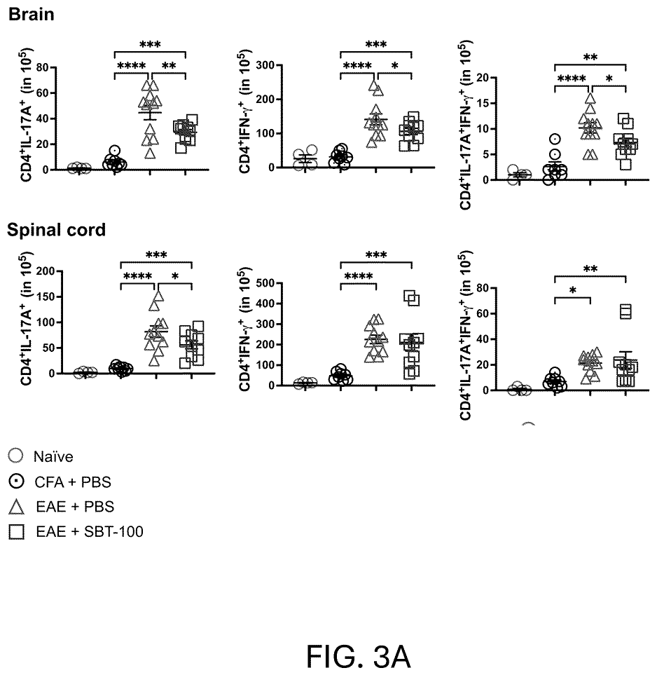

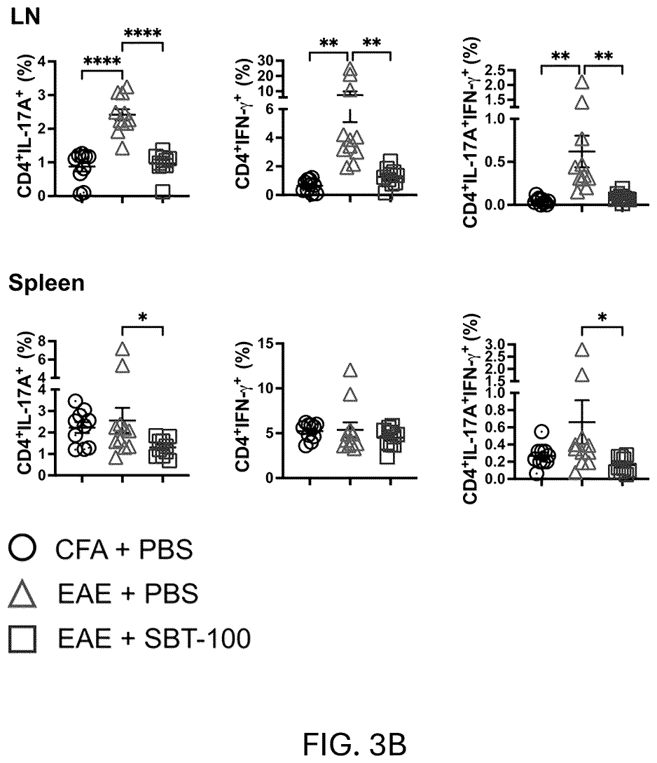

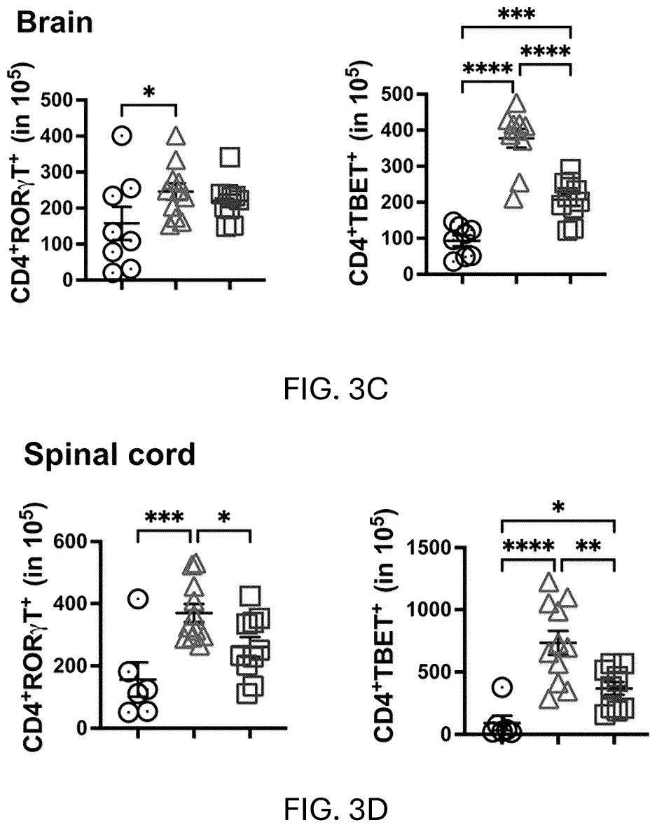

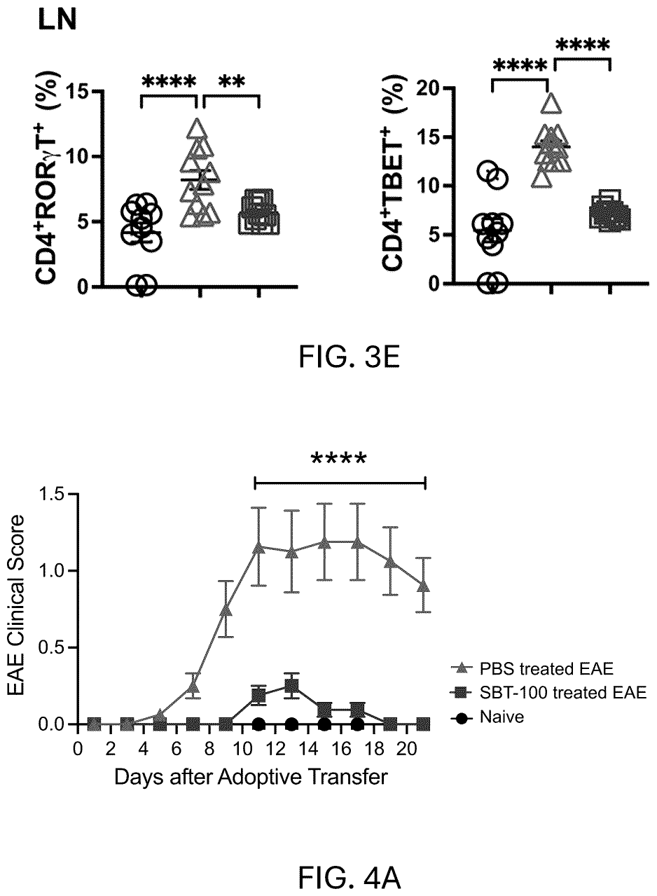

Significant infiltration of T cells secreting IL-17A and/or IFN-γ in the brain and spinal cord correlated with the severe EAE in the untreated mice. Flow cytometry was done to assay the percentage of T cells expressing IL-17 and/or IFN-γ in the LN or spleen. As shown in cytometry percentage bar graphs, T cells expressing IL-17 and/or IFN-γ were suppressed in the LN or spleen of mice treated with SBT-100 (FIG. 3A). There was an increase of T cells secreting IL-17A and/or IFN-γ in the spleen and lymph nodes of these mice (FIG. 3B). In contrast, a significant decrease in T cells secreting IL-17A and/or IFN-γ was observed in the SBT-100 treated mice (FIG. 3A, 3B). Cytometry percentage bar graphs showing expression of ROR-γt or T-bet in the brain, spinal cord and lymph node are shown in FIGS. 3C-3E. Data represent at least 2 independent experiments and presented as mean±SEM. ((*p<0.05; **p<0.01; ***p<0.001; ****p<0.0001). The decrease in Th1 and Th17 in these tissues correlated with corresponding decrease in the levels of ROR-γt and T-bet which are Th17 and Th1 lineage-specifying transcription factors (FIGS. 3C-3E and data not shown). Taken together, these results show that SBT-100 mitigates EAE by inhibiting STAT1 and STAT3 pathways required for Th1 and Th17 development and inducing inflammation in the spinal cord and brain, respectively.

### Example 4. Lymphocytes from SBT-100 Treated EAE Mice have Reduced Capacity to Transfer EAE

Although IL-17 and IFN-γ mediate EAE in the brain and spinal cord, granulocyte macrophage colony-stimulating factor (GM-CSF) knockout mice are resistant to EAE, suggesting that GM-CSF also plays critical role in the development of EAE. GM-CSF is produced by a wide variety of cell types, upregulates expression of MHC class II and proinflammatory cytokine by microglia, macrophages, DCs and promotes the differentiation of CD4⁺ T cells into effector T-cell subsets. To rule out the possibility that the suppression of EAE observed was derived from SBT-100 effects on antigen-presenting myeloid cells (APCs), it was determined whether transfer of MOG-specific encephalitogenic CD4⁺ T cells into EAE mice would attenuate the disease.

For adoptive transfer experiments, EAE was induced in wild-type C57BL/6 mice by active immunization with MOG₃₅₋₅₅ and treated with PBS or SBT-100 as described in Example 2. Mice exhibiting clinical features of EAE were sacrificed, and cells isolated from lymph nodes and spleens were reactivated for 72 hours in the presence of MOG35-55 (20 μg/ml) at 6×10⁶/ml. Cells were washed, resuspended in medium and 5×10⁷ cells. EAE was then induced in naive syngeneic mice and by day 9 post-immunization, when EAE was established, the MBP-specific encephalitogenic T cells were adoptively transferred. To demonstrate that SBT-100 can suppress established EAE, some of the mice were treated with SBT-100 beginning on day 12 after adoptive transfer until day 21 post-adoptive transfer. Mice were monitored daily for EAE disease symptoms and scored as described above.

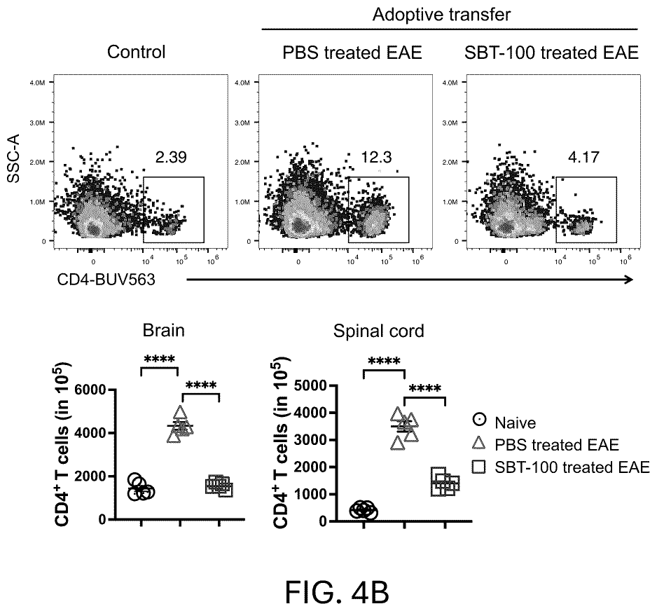

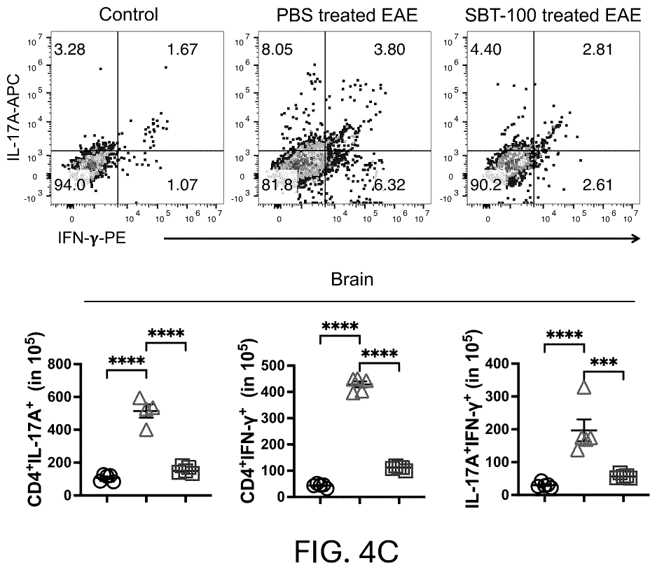

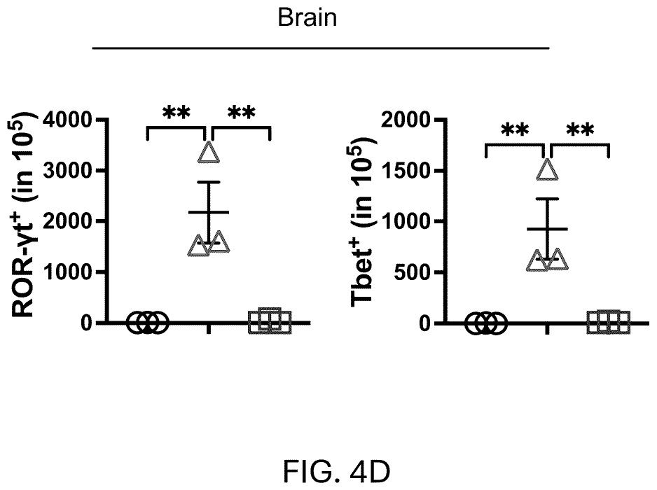

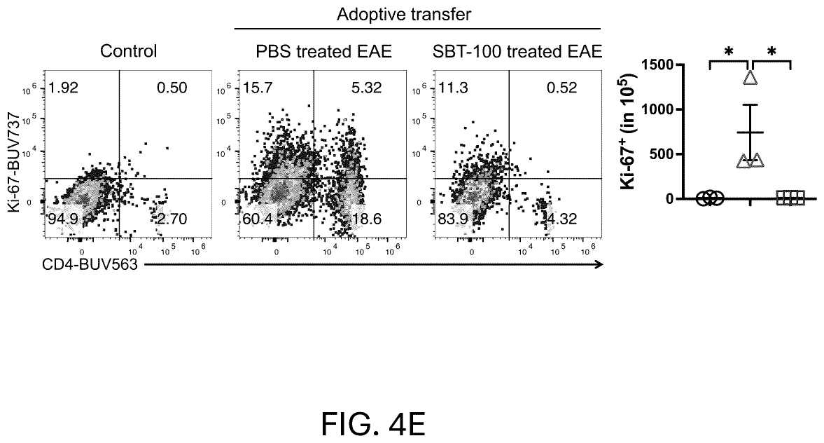

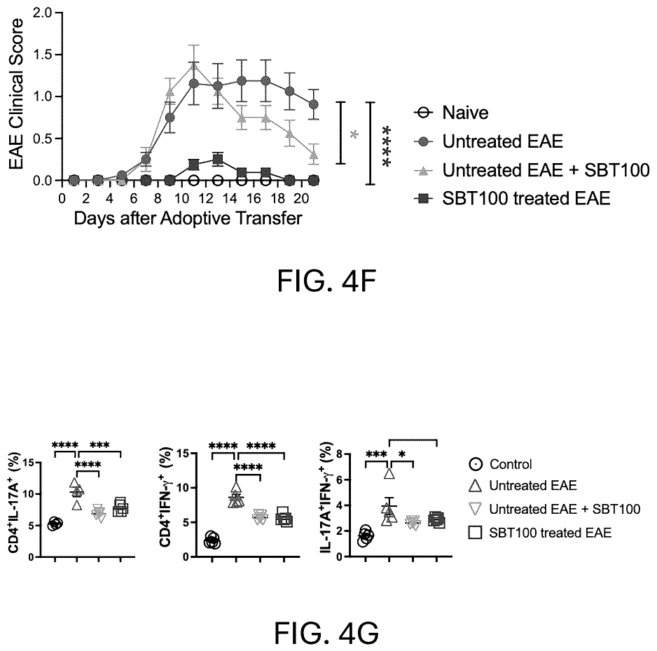

EAE Clinical score of naive mice, mice that received cells from untreated EAE mice, or SBT-100 treated EAE mice are shown in FIG. 4A (n=8). Mice that received cells from the untreated mice developed severe EAE, while mice that received cells from SBT-100 treated mice developed mild EAE with delayed onset. CD4⁺ T cells were isolated from the brain, spinal cord, spleen or lymph nodes of the mice (gating strategy is shown in FIG. 2B). Analysis of the cells from mice that received SBT-100 encephalitogenic showed significant correlation of EAE attenuation and decrease in the numbers of CD4⁺ T cells that infiltrated the brain and spinal cord (FIG. 4B). Intracellular cytokine staining and FACS analysis were done to show the frequency of IL-17A+, IFN-γ+ and IL-17A+IFN-γ+ double positive cells in the brain or spinal cord. Concomitant reduction of Th17 and Th1 cells secreting IL-17 and/or IFN-γ (FIG. 4C) or the transcription factors ROR-γt and T-bet in the brain and spinal cord was observed (FIG. 4D). The decrease in inflammatory CD4⁺ T cells correlated with significant reduction of lymphocyte proliferative capacity in mice adoptively transferred with cells from mice treated with SBT-100 (FIG. 4E). To directly demonstrate that SBT-100 can suppress established EAE, a subset of adoptive transfer EAE mice were treated with SBT-100 from Day 12 to Day 21 post adoptive transfer. Consistent with disease suppression in the active immunization model, SBT-100 ameliorated established EAE and also inhibited the expansion of Th17 and Th1 (FIGS. 4F and 4G) (*p<0.05; **p<0.01; ***p<0.001; ****p<0.0001).

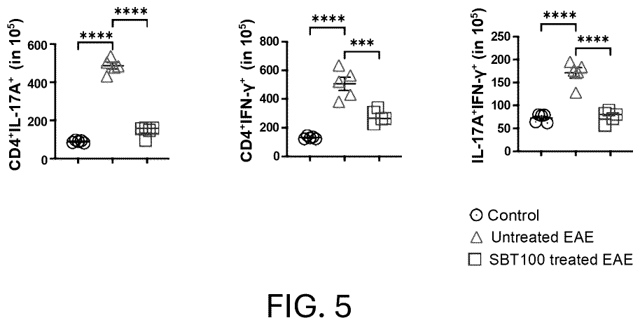

Additionally, SBT-100 suppress Th17 and Th1 cells infiltration of the CNS. Frequency plot of intracellular cytokine staining and FACS analysis showing the number of IL17A+, IFN-γ+, IL-17A+IFN-γ+ double positive cells in spinal cord. (FIG. 5) Data represent at least 2 independent experiments and presented as mean±SEM. (***p<0.001; ****p<0.0001).

Although the present invention has been described in considerable detail with reference to certain preferred embodiments, other embodiments are possible. The steps disclosed for the present methods, for example, are not intended to be limiting nor are they intended to indicate that each step is necessarily essential to the method but instead are exemplary steps only. Therefore, the scope of the appended claim should not be limited to the description of preferred embodiments contained in this disclosure. All references cited herein are incorporated by reference in their entirety.

Insofar as the description above discloses any additional subject matter that is not within the scope of the claims below, the inventions are not dedicated to the public and the right to file one or more applications to claim such additional inventions is reserved.

What is claimed is:

1. A method of treating neuroinflammation in a subject in need thereof, comprising administering to the subject an effective amount of a single-domain antibody (sdAb) comprising SEQ ID NO:1.

2. The method of claim 1, wherein the neuroinflammation is multiple sclerosis.

3. The method of claim 2, wherein the subject has secondary progressive multiple sclerosis or primary progressive multiple sclerosis.

4. The method of claim 1, wherein the subject is a human patient diagnosed with relapsing-remitting multiple sclerosis.

5. The method of claim 1, wherein administration is by intravenous, subcutaneous, or intrathecal injection.

6. The method of claim 1, wherein the effective amount is from 0.1 to 20 mg/kg body weight per dose.

7. The method of claim 1, wherein the effective amount is administered in multiple doses over a period of 7 to 28 days.

8. The method of claim 1, further comprising administering a pharmaceutically acceptable excipient selected from the group consisting of mannitol, sucrose, and polysorbate 80.

9. The method of claim 2, further comprising co-administering a disease-modifying therapy for multiple sclerosis.

10. The method of claim 9, wherein the therapy for multiple sclerosis is selected from the group consisting of interferon-β, glatiramer acetate, fingolimod and ocrelizumab.

11. A method of preventing neuroinflammation in a subject in need thereof, comprising administering to the subject an effective amount of a sdAb comprising SEQ ID NO:1.

12. The method of claim 11, wherein the neuroinflammation is multiple sclerosis.

13. The method of claim 12, wherein the subject has secondary progressive multiple sclerosis or primary progressive multiple sclerosis.

14. The method of claim 11, wherein the subject is a human patient diagnosed with relapsing-remitting multiple sclerosis.

15. The method of claim 11, wherein administration is by intravenous, subcutaneous, or intrathecal injection.

16. The method of claim 11, wherein the effective amount is from 0.1 to 20 mg/kg body weight per dose.

17. The method of claim 11, wherein the effective amount is from 0.5 mg/kg to 5 mg/kg administered once daily for 10 days.

18. The method of claim 11, further comprising administering a pharmaceutically acceptable excipient selected from the group consisting of mannitol, sucrose, and polysorbate 80.

19. The method of claim 12, further comprising co-administering a disease-modifying therapy for multiple sclerosis.

20. The method of claim 19, wherein the therapy for multiple sclerosis is selected from the group consisting of interferon-β, glatiramer acetate, fingolimod and ocrelizumab.
